(19) United States
(12) Patent Application Publication
(10) Pub. No.: US 2009/0138037 A1
(43) Pub. Date: May 28, 2009

(54) STRAIN LIMITING TETHER FOR A CATHETER

(75) Inventor: Patrick Griffin, Castlegar (IE)

Correspondence Address: Sughrue Mion-ABBOTT LABS, 2100 Pennsylvania Avenue, N.W., Washington, DC, 20037, US

(73) Assignee: SALVIAC LIMITED, Dublin (IE)

(21) Appl. No.: 12/256,544

(22) Filed: Oct. 23, 2008

Related U.S. Application Data
(60) Provisional application No. 60/983,178, filed on Oct. 27, 2007.

(51) Int. Cl. A61M 29/02

(52) U.S. Cl. 606/200

(57) ABSTRACT

A catheter tip and a catheter system are provided. The catheter tip includes a shaft which comprises at least two segments at a distal end portion thereof, wherein the at least two segments define a reception space for receiving a filter element; an expandable tip body; and at least two tethers connecting the segments. The catheter system includes a guide catheter; and a retrieval catheter for insertion into the guide catheter, the retrieval catheter having a catheter tip including a shaft which comprises at least two segments at a distal end portion thereof, wherein the at least two segments define a reception space for receiving a filter element; an expandable tip body; and at least two tethers connecting the segments.

### BACKGROUND OF THE INVENTION

1. Field of the Invention

The present invention relates to catheters, and more particularly, to a catheter which includes a strain limiter to prevent the possibility of bursting of the catheter upon loading or retrieving a filter element and to compress plaque present in the filter element.

2. Description of the Related Art

Catheters according to the related art are inserted into a body passageway of a patient, advanced through the body passageway to a treatment area of the patient, and used to deposit or retrieve an object such as a filter element, balloon, or stent in the general proximity of the treatment area.

Related art catheters present a number of problems. Generally the object to be loaded into the catheter has a larger size than the catheter tip. For example, typically a filter element is deployed in the body passageway in order to filter any plaque or other particulate matter that is dislodged from the treatment area during the treatment process. Thus, the filter element must be able to expand to the same dimensions of the body passageway in order to provide effective filtering to prevent plaque or particulate matter from passing by the filter without being trapped in the filter. Once treatment is accomplished, the filter, which includes the filtered plaque as a payload, is removed from the body passageway. Thus, the relatively large object, i.e., the filter element, must enter into a relatively small catheter.

When the large object is brought into the catheter, the tip of the catheter is prone to buckling due to the large compressive forces acting on the tip of the catheter as the large object is pulled in to the catheter.

In order to address this problem, U.S. Patent Application Publication No. 2006/0293696 discloses a retrieval catheter including reinforcement columns. The reinforcement columns provide additional stiffening to the catheter tip in order to prevent buckling. The disclosure of U.S. Patent Application Publication No. 2006/0293696 is incorporated herein by reference in its entirety.

However, related art catheters suffer from a number of problems. In related art catheters, the expansion of the tip b depends upon the soft expandable material of the tip body. In particular, the outer diameter of the catheter tip depends on a combination of the stress-strain behavior and geometry of the material of the tip body. Thus, in some cases, the stress and strain caused by the filter element will cause the catheter to burst. Moreover, the plaque material can often be large and hard, and therefore is not easily compressed by the filter. This exerts additional pressure on the catheter as the filter is retrieved into the tip, often resulting in a bursting or rupturing of the catheter.

### SUMMARY OF THE INVENTION

Exemplary embodiments of the present invention address the above disadvantages and other disadvantages not described above. Also, the present invention is not required to overcome the disadvantages described above, and an exemplary embodiment of the present invention may not overcome any of the problems described above.

An object of the present invention is to limit the expansion of the catheter when accepting a filter element or other device, thus preventing the catheter from bursting due to the strain of accepting the payload.

Another object of the present invention is to more easily compress any plaque or other particulate material that is present in the filter element, if needed, so that the plaque may be more easily harvested and removed.

According to an aspect of the present invention, there is provided a catheter tip including a shaft which comprises at least two segments at a distal end portion thereof, wherein the at least two segments define a reception space for receiving a filter element; an expandable tip body; and at least two tethers connecting the segments.

According to another aspect of the present invention, there is provided a catheter tip including a shaft which comprises at least two segments at a distal end portion thereof, wherein the at least two segments define a reception space for accepting a filter element; an expandable tip body; and means, connecting the at least two segments, for limiting an expansion of the catheter tip during retrieval of a filter element into the reception space.

According to yet another aspect of the present invention, there is provided a catheter system including a guide catheter; and a retrieval catheter for insertion into the guide catheter, the retrieval catheter having a catheter tip including a shaft which comprises at least two segments at a distal end portion thereof, wherein the at least two segments define a reception space for receiving a filter element; an expandable tip body; and at least two tethers connecting the segments

### BRIEF DESCRIPTION OF THE DRAWINGS

The above and other aspects of the present invention will become more apparent by describing in detail exemplary embodiments thereof with reference to the attached drawings, in which:

FIG. 1 is a perspective view of a distal end of a catheter according to an exemplary embodiment of the present invention;

FIG. 2 is another perspective view of the distal end of the catheter of FIG. 1;

FIG. 3 is a perspective view of a distal end of a catheter shaft of the catheter of FIGS. 1 and 2 with reinforcement elements and tether elements according to an exemplary embodiment of the present invention;

FIGS. 4A and 4B are perspective views showing a close-up view of a distal end of a catheter according to an exemplary embodiment of the present invention;

FIG. 5A is a cross sectional side view of the catheter of FIGS. 1-3;

FIG. 5B is a side view of a filter element;

FIG. 5C is a partially cross-sectional side view of the filter of FIG. 4B collapsed inside of the catheter of FIG. 5A;

FIGS. 5D and 5E are cross-sectional side views of the catheter and the collapsed filter, respectively, of FIG. 5C;

FIG. 5F is a side view of a filter element and a catheter of FIGS. 1-3;

FIG. 5G is a side view of the filter element of FIG. 5F collapsed inside the catheter of FIG. 5F;

FIGS. 6A and 6B are a cross-sectional side view and a head-on view, respectively, of an unexpanded catheter according to an exemplary embodiment of the present invention; and

FIGS. 7A and 7B are a cross-sectional side view and a head-on view, respectively, of an expanded catheter according to an exemplary embodiment of the present invention.

### DESCRIPTION OF EXEMPLARY EMBODIMENTS OF THE PRESENT INVENTION

Hereinafter, aspects of the present inventive concept will be described in detail with reference to the drawings.

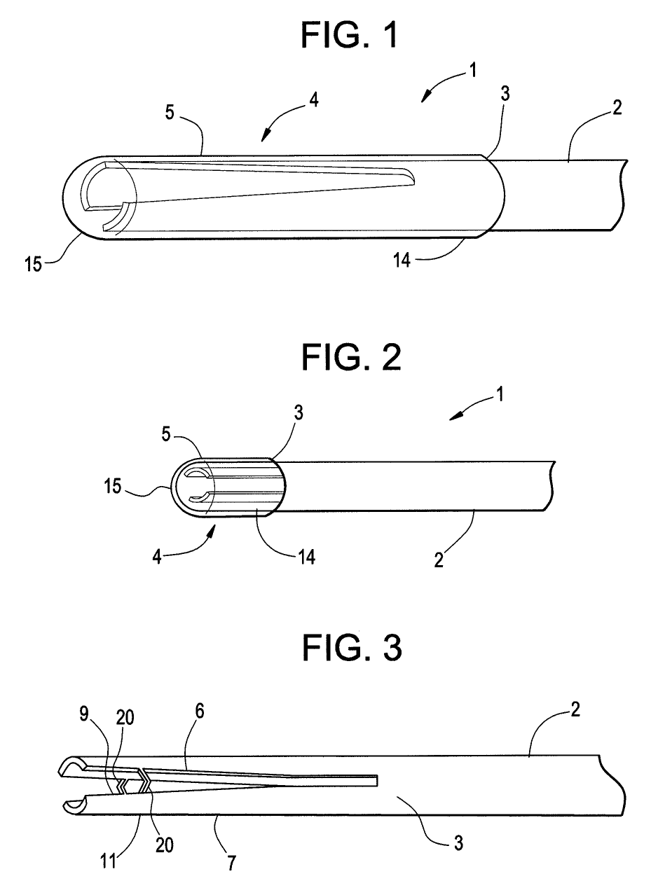

Referring to FIGS. 1-3, a catheter 1 according to an exemplary embodiment of the present invention includes a shaft 2 having a proximal end of the shaft 2 and a distal end 3 of the shaft 2, and a catheter tip 4 at the distal end 3 of the shaft 2. The tip 4 includes an expandable tip body 5 and at least two segments 6, 7 extending along part of the length of the tip body 5, and are provided, for example, in the form of reinforcement splines.

The segments 6, 7 are arcuate and spaced-apart circumferentially around the tip 4, and are located radially opposing one another.

The segments 6, 7 are embedded within the tip body 5, with the tip body 5 extending over the entire outer surface of the segments 6, 7 and over the entire inner surface of the segments 6, 7.

The tip body 5 may also extend within the longitudinal space between laterally adjacent segments 6, 7 to form soft segment cut-outs, and is fixedly attached to the segments 6, 7, for example, by heat-sealing.

The distal end 15 of the tip body 5 extends distally of the distal end 12 of the segments 6, 7, and the distal end 15 of the tip body 5 is rounded. In this manner, the tip 4 presents a smooth crossing profile to facilitate passage of the catheter 1 through the body passageway.

The tip body 5 has a reception space 9 extending therethrough from a proximal end 14 of the tip body 5 to a distal end 15 of the tip body 5. The reception space 9 facilitates retrieval of an object, such as a filter element, into the tip body 5.

The at least two segments 6, 7 are stiff relative to the tip body 5 and act to reinforce the tip body 5, especially during insertion or reception of an object into the reception space 9, to minimize the possibility of buckling or collapse of the tip 4. However, segments 6, 7 also maintain sufficient flexibility to enable passage of the tip 4 through potentially narrow and/or tortuous body passageways.

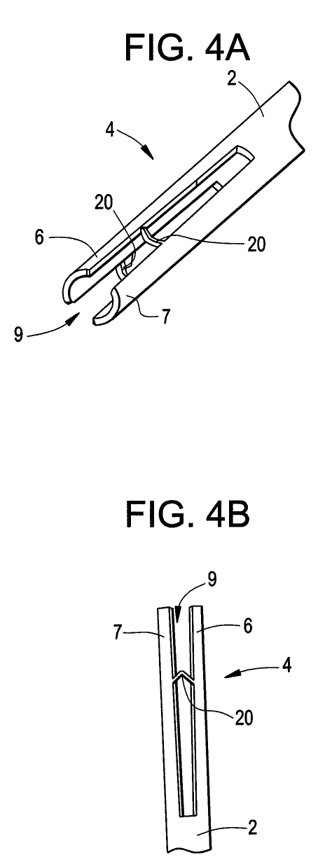

FIGS. 4A and 4B show a close-up perspective view of the tip 4. The tip 4 has one or more tethers 20 added to each soft segment cut-out. For example, FIGS. 4A and 4B show a single tether 20 placed on opposite sides of the reception space 9. The tethers 20 are formed as an integral part of the material of segments 6, 7 and are cut at the same time as the segments 6, 7. The tethers 20 are provided with enough length to provide some slack.

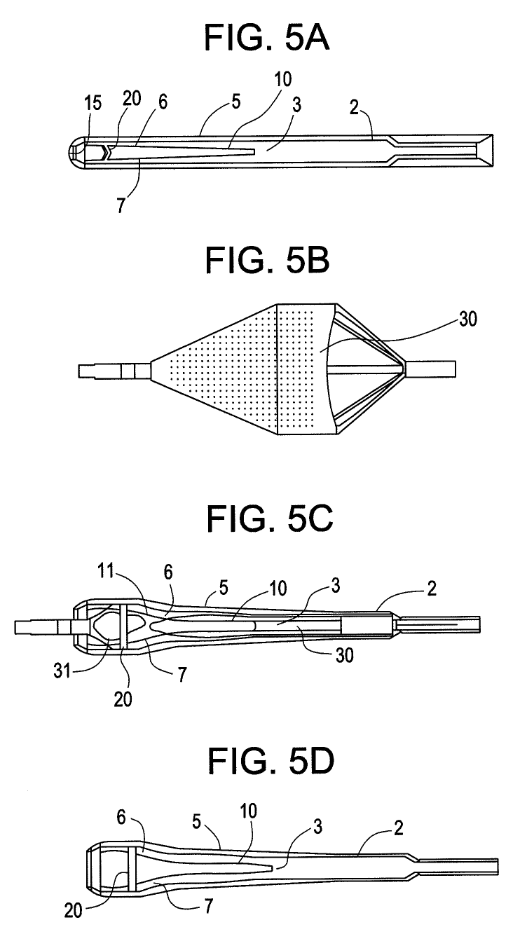

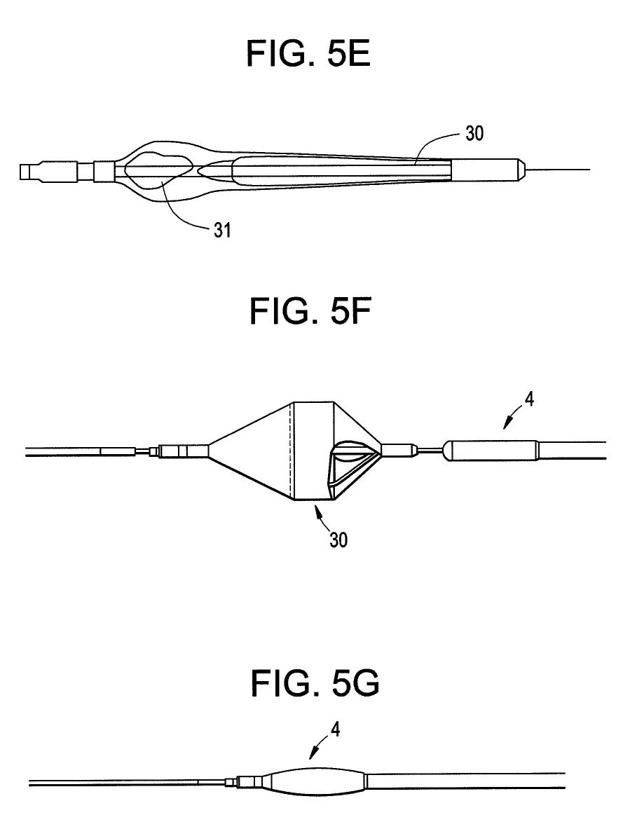

FIGS. 5A-5G show the catheter 1 in use. With reference now to FIGS. 5A-5G, the catheter 1 is inserted into a body passageway, for example, a vasculature, and advanced through the vasculature until the tip 4 is proximally adjacent of an object to be retrieved, such as a filter element 30 with captured plaque and/or particulate material 31 therein. The tip 4 is then moved distally relative to the filter element 30 to retrieve at least part of the filter element 30 along with the captured plaque 31 into the tip 4. The tip body 5 expands radially outwardly to accommodate the relatively large filter element 30 into the tip 4 during retrieval, as shown in FIG. 4C. Examples of filter elements are disclosed in U.S. Pat. No. 6,336,934, the entire disclosure of which is incorporated herein by reference.

During expansion of the tip 4, the distal part 11 of the segments 6, 7 acts a lead-in or guide for the filter element 30 being retrieved, and the proximal part 10 of the segments 6, 7 acts a stiffness transition. The gap between the segments 6, 7 can expand until the slack in the tethers 20 is eliminated and the tethers 20 become taut, as shown in FIG. 5C. Thus, the tip 4 expands and the slack of the tethers 20 allows the expansion up to the point where the tethers 20 become taut. At this point, the force to extend the tip 4 is no longer just dependent on the expansion of the soft segment, but also on the force to extend the tensioned tethers 20, and the tethers 20 serve as strain limiters and act to retrain further radial expansion of the tip 4.

As the tip 4 expands and the filter element 30 is retrieved, a support frame 36 of the filter element 30 collapses down under the restraining force of the tethers 20, as illustrated in FIGS. 4C to 4E.

The tethers 20 also act in conjunction with the proximal part 10 of the segments 6, 7 to compress the captured plaque 31, and provide a more uniform strain zone which thus provides constant radial force on the retrieved filter element 30.

When the filter element 30 has been fully retrieved into the tip 4, the retrieval catheter 1 is withdrawn from the vasculature.

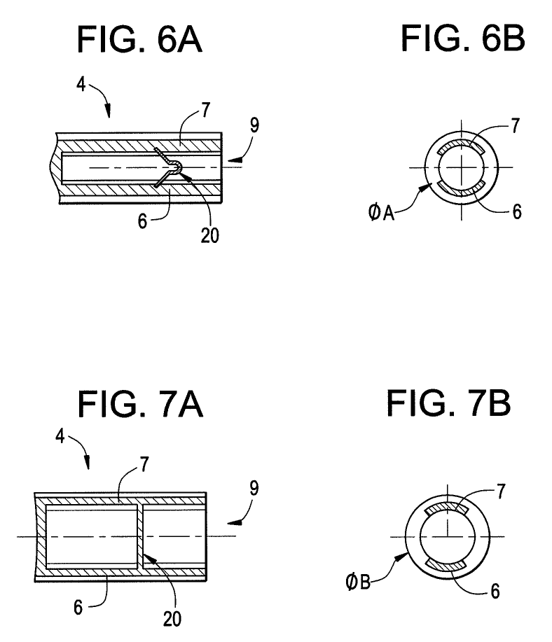

FIGS. 6A and 6B and 7A and 7B further illustrate the restraint on the radial expansion of the tip 4. In FIGS. 6A-6B and 7A-7B, the segments 6, 7 are shown as hatched.

FIG. 6A shows the distal end of the tip 4 including the segments 6, 7 and the tether 20 in a normal slack position. In the slack position, the opening diameter of the tip is A, as shown in FIG. 6B.

FIG. 7A shows the distal end of the tip 4 including the segments 6, 7 and the tether 20 in a expanded position in which the tethers 20 are taut. In the expanded position, the opening diameter of the tip is B.

The length of the tethers 20 available effectively determines the maximum expansion of the tip 4, as may be seen from an examination of FIGS. 6A and 6B and 7A and 7B. The length of the tethers 20 are selected so that when the tethers 20 are extended and the slack is removed, the overall expanded outer diameter B of the tip 4 is smaller than a guide catheter or sheath inner diameter. It will be understood that the actual geometry of the tethers 20 is not critical to the strain relief function of the tethers 20.

Thus, the final tip 4 outer diameter post retrieval is limited by the expansion of the tethers 20, whereas the final tip outer diameter on a related art catheter tip depends on a combination of the stress-strain behavior and geometry of the soft segment material.

The foregoing exemplary embodiments and advantages are merely exemplary and are not to be construed as limiting the present invention. The present teaching can be readily applied to other types of apparatuses. Also, the description of the exemplary embodiments of the present invention is intended to be illustrative, and not to limit the scope of the claims, and many alternatives, modifications, and variations will be apparent to those skilled in the art.

What is claimed is:

1. A catheter tip comprising:
a shaft which comprises at least two segments at a distal end portion thereof, wherein the at least two segments define a reception space for receiving a filter element;
an expandable tip body; and
at least two tethers connecting the segments.

2. The catheter tip according to claim 1, wherein the at least two segments are arcuate splines and are disposed on radially opposite sides of the shaft.

3. The catheter tip according to claim 1, wherein the at least two tethers are provided with a length of slack when the catheter tip is in an unexpanded position in which the filter element is not present in the reception space but the at least two tethers become taut during retrieval of the filter element into the reception space.

4. The catheter tip according to claim 1, wherein the at least two tethers are provided with a length of slack such that the at least two tethers experience no tension when a filter element is not present in the reception space but the at least two tethers experience tension during retrieval of the filter element into the reception space.

5. The catheter tip according to claim 1, wherein the at least two segments are formed of a material harder in stiffness than a material of the expandable tip body.

6. The catheter tip according to claim 1, wherein the at least two segments are splines.

7. A catheter tip comprising:
a shaft which comprises at least two segments at a distal end portion thereof, wherein the at least two segments define a reception space for accepting a filter element;
an expandable tip body; and
means, connecting the at least two segments, for limiting an expansion of the catheter tip during retrieval of a filter element into the reception space.

8. The catheter tip according to claim 7, wherein the means limits a strain between the at least two segments during the retrieval of the filter element into the reception space.

9. The catheter tip according to claim 7, wherein the means limits a strain as the tip body expands to accommodate the filter element into the reception space.

10. A catheter system comprising:
a guide catheter; and
a retrieval catheter for insertion into the guide catheter, the retrieval catheter having a catheter tip comprising:
a shaft which comprises at least two segments at a distal end portion thereof, wherein the at least two segments define a reception space for receiving a filter element;
an expandable tip body; and
at least two tethers connecting the segments.

11. The catheter system according to claim 10, wherein the guide catheter has an inner diameter, and a length of the at least two tethers is such that, during retrieval of the filter element when the at least two tethers are fully taut, an outer diameter of the retrieval catheter is smaller than the inner diameter of the guide catheter.
